SEQUENCE LISTING (4 sequences)

SEQ ID NO: 1 - DNA, 1361 bp, Mus musculus
  CDS (48)...(1064)
tcttttttttttttcattgcagaactgagattgcaccactcctgaaaatggacttagtcatccaagactggaccattaatattacagcactgaaagaaagcaatgacaatggaatatcattttgtgaagttgtgtctcgtaccatgacttttctttccctcatcattgccttagttgggctggttggaaatgccacagtgttatggtttctgggcttccagatgagcaggaatgccttctctgtctacatcctcaaccttgctggtgctgactttgtcttcatgtgctttcaaattgtacattgtttttatattatcttagacatctacttcatccccactaattttttttcatcttacactatggtgttaaacattgcttaccttagtggtctgagcatcctcactgtcattagcactgaacgcttcctatctgtcatgtggcccatctggtaccgctgccaacgcccaaggcacacatcagctgtcatatgtactgtgctttgggtcttgtccctggtgttgagcctcctggaaggaaaggaatgtggcttcctatattacactagtggccctggtttgtgtaagacatttgatttaatcactactgcatggttaattgttttatttgtggttctcttgggatccagtctggccttggtgcttaccatcttctgtggcttacacaaggttcctgtgaccaggttgtatgtgaccattgtgtttacagtgcttgtcttcctgatctttggtctgccctatgggatctactggttcctcttagagtggattagggaatttcatgataataaaccttgtggttttcgtaacgtgacaatatttctgtcctgtattaacagctgtgccaaccccatcatttacttccttgttggctccattaggcaccatcggtttcaacggaagactctcaagcttcttctgcagagagccatgcaagactctcctgaggaggaagaatgtggagagatgggttcctcaagaagacctagagaaataaaaactgtctggaagggactgagagctgctttgatcaggcataaatagctttgaagagaactatgtttttatcactttgtggcattttcataatgttgtttagttgatgacccaaggttaactcagttggggaagtagtcaatgttgtagaagttgattgatattgaacttgttataaatactgagtacagtatttttgcagctatcttgctcagagctttaccaactccatttgatgggactccttataagctctatggggtccaggagaggtgttgaccacaattgacaaatccctcttcagaagaaaactcaagaaagtgcaatgaaaagttatatttcttt

SEQ ID NO: 2 - protein, 338 aa, Mus musculus
MDLVIQDWTINITALKESNDNGISFCEVVSRTMTFLSLIIALVGLVGNATVLWFLGFQMSRNAFSVYILNLAGADFVFMCFQIVHCFYIILDIYFIPTNFFSSYTMVLNIAYLSGLSILTVISTERFLSVMWPIWYRCQRPRHTSAVICTVLWVLSLVLSLLEGKECGFLYYTSGPGLCKTFDLITTAWLIVLFVVLLGSSLALVLTIFCGLHKVPVTRLYVTIVFTVLVFLIFGLPYGIYWFLLEWIREFHDNKPCGFRNVTIFLSCINSCANPIIYFLVGSIRHHRFQRKTLKLLLQRAMQDSPEEEECGEMGSSRRPREIKTVWKGLRAALIRHK

SEQ ID NO: 3 - DNA, 1300 bp, Homo sapiens
  CDS (171)...(1160)
tccctggcccttaataaatgacttaatctcttcaagcctctgatttcctctcctgtaaaacaggggcggtaattaccacataacaggctggtcatgaaaatcagtgaacatgcagcaggtgctcaagtcttgtttttgtttccaggggcaccagtggaggttttctgagcatggatccaaccaccccggcctggggaacagaaagtacaacagtgaatggaaatgaccaagcccttcttctgctttgtggcaaggagaccctgatcccggtcttcctgatccttttcattgccctggtcgggctggtaggaaacgggtttgtgctctggctcctgggcttccgcatgcgcaggaacgccttctctgtctacgtcctcagcctggccggggccgacttcctcttcctctgcttccagattataaattgcctggtgtacctcagtaacttcttctgttccatctccatcaatttccctagcttcttcaccactgtgatgacctgtgcctaccttgcaggcctgagcatgctgagcaccgtcagcaccgagcgctgcctgtccgtcctgtggcccatctggtatcgctgccgccgccccagacacctgtcagcggtcgtgtgtgtcctgctctgggccctgtccctactgctgagcatcttggaagggaagttctgtggcttcttatttagtgatggtgactctggttggtgtcagacatttgatttcatcactgcagcgtggctgatttttttattcatggttctctgtgggtccagtctggccctgctggtcaggatcctctgtggctccaggggtctgccactgaccaggctgtacctgaccatcctgctcacagtgctggtgttcctcctctgcggcctgccctttggcattcagtggttcctaatattatggatctggaaggattctgatgtcttattttgtcatattcatccagtttcagttgtcctgtcatctcttaacagcagtgccaaccccatcatttacttcttcgtgggctcttttaggaagcagtggcggctgcagcagccgatcctcaagctggctctccagagggctctgcaggacattgctgaggtggatcacagtgaaggatgcttccgtcagggcaccccggagatgtcgagaagcagtctggtgtagagatggacagcctctacttccatcagatatatgtggctttgagaggcaactttgcccctgtctgtctgatttgctgaactttctcagtcctgattttaaaacagttaagagagtccttgtgaggattaagtgagaca

SEQ ID NO: 4 - protein, 330 aa, Homo sapiens
MDPTTPAWGTESTTVNGNDQALLLLCGKETLIPVFLILFIALVGLVGNGFVLWLLGFRMRRNAFSVYVLSLAGADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDHSEGCFRQGTPEMSRSSLV

(19) United States
(12) Patent Application Publication
(10) Pub. No.: US 2008/0014586 A1
(43) Pub. Date: Jan. 17, 2008

(54) IDENTIFICATION OF A RECEPTOR CONTROLLING MIGRATION AND METASTASIS OF SKIN CANCER CELLS

(75) Inventors: Mark Zylka, Pasadena, CA (US); Xinzhong Dong, Pasadena, CA (US); David J. Anderson, Altadena, CA (US)

Correspondence Address: KNOBBE MARTENS OLSON & BEAR LLP, 2040 MAIN STREET, FOURTEENTH FLOOR, IRVINE, CA, 92614, US

(73) Assignee: California Institute of Technology, Pasadena, CA (US)

(21) Appl. No.: 11/777,907

(22) Filed: Jul. 13, 2007

Related U.S. Application Data
(62) Division of application No. 10/601,305, filed on Jun. 19, 2003.
(60) Provisional application No. 60/391,127, filed on Jun. 21, 2002.

(51) Int. Cl. C12Q 1/68

(52) U.S. Cl. 435/6

(57) ABSTRACT

The invention relates generally to genes expressed in skin cancer cells, particularly melanoma tumor cells, and their role in migration and metastasis. Methods for identifying melanoma cells are provided, as are methods of treating melanoma. Methods for identifying compounds that are useful in the treatment of melanoma are also provided.

### REFERENCE TO RELATED APPLICATIONS

This application is a divisional of U.S. application Ser. No. 10/601,305, filed Jun. 19, 2003, which claims priority under 35 U.S.C. §119(e) to U.S. Provisional Application 60/391,127, filed Jun. 21, 2002, which is incorporated by reference in its entirety.

### BACKGROUND OF THE INVENTION

1. Field of the Invention

The invention relates generally to genes expressed in melanoma tumor cells. Methods for identifying melanoma tumor cells, for treating melanoma and for identifying compounds that may be used to treat melanoma are provided.

2. Description of the Related Art

A family of orphan G protein-coupled receptors known as mas-related genes (MRGs) has recently been identified (see, e.g., U.S. patent application Ser. Nos. 09/704,707 and 09/849,869, Dong et al. Cell 106:619-632 (2001), incorporated herein by reference). The Mrg family of GPCRs comprises three major subfamilies (MrgA, B and C). The MrgA subfamily consists of at least twenty members in mice. Four human receptors that are most closely related to the MrgA subfamily have also been identified: MrgX1; MrgX2 (SEQ ID NO: 4); MrgX3; and MrgX4.

The MrgB subfamily consists of at least twelve members in mice: MrgB1 (SEQ ID NO: 2); MrgB2; MrgB3; MrgB4; MrgB5; MrgB6; MrgB7; MrgB8; MrgB9; MrgB10; MrgB11; and MrgB12. In rat there are two subfamilies of MrgB-related genes. One of these subfamilies is most closely related to murine MrgB1. The other subfamily is most closely related to murine MrgB4 and MrgB5 (FIG. 1).

The Mrg family members that were originally characterized were all found to be specifically expressed in primary sensory neurons of the dorsal root ganglia (DRG).

Members of the Mrg family of receptors appear to be activated by neuropeptide ligands comprising RFamide and RYamide C-termini, based on ligand screens in heterologous cell expression systems (Dong et al., supra; Han et al. Proc. Natl. Acad. Sci. USA 99:14740-14745 (2002)).

The RFamide ligand, KiSS has been identified as an inhibitor of migration and metastasis in melanoma cells (see, e.g., Ohtaki et al. Nature 411:613-617 (2001)). Consistently, it was determined that KiSS is able to activate some Mrgs when they are expressed in heterologous cells. Thus, activation of Mrgs in melanoma cells may inhibit metastasis and migration and drugs that bind to a subset of Mrgs expressed that has now been found to be expressed in melanoma cells may provide new therapies for treating metastatic melanoma, one of the most dangerous cancers known.

### SUMMARY OF THE INVENTION

In one aspect of the invention, a method of diagnosing skin cancer, preferably melanoma, in a patient is provided. A tissue sample, preferably a skin sample, is obtained from a patient suspected of suffering from melanoma, or at risk of suffering from melanoma. For example, the skin sample may be a mole or skin lesion that a physician believes may be melanoma. It is determined whether cells in the tissue sample express MrgX2. Expression of MrgX2 indicates that the patient is suffering from melanoma. Preferably, MrgX2 has the amino acid sequence of SEQ ID NO:4.

In one embodiment MrgX2 expression is determined by contacting the tissue sample with an antibody that specifically binds MrgX2 and determining if the antibody binds to the tissue sample. The tissue sample may be treated prior to contacting it with the antibody as necessary to allow the antibody to contact the cells. The antibody is preferably detectably labeled, for example with a radioactive label or a fluorescent label. In a preferred embodiment, the antibody is a monoclonal antibody.

In another embodiment MrgX2 expression is determined by contacting the tissue sample with a nucleic acid probe that is complementary to a portion of the MrgX2 nucleic acid of SEQ ID NO: 3 and determining if the probe binds to the tissue sample. Preferably the probe is detectably labeled, for example with a radioactive label or a fluorescent label.

According to another aspect of the invention, a method of diagnosing melanoma in a patient comprises obtaining a tissue sample from a patient suspected of suffering from melanoma. Preferably the tissue sample comprises skin cells. RNA, is prepared from the tissue sample and contacted with a nucleotide probe that is capable of hybridizing to the MrgX2 nucleotide sequence of SEQ ID NO: 3 under stringent conditions. Specific binding of the probe indicates that the sample comprises melanoma and the patient is diagnosed as suffering from melanoma. In one embodiment mRNA is prepared from the tissue sample. In another embodiment total RNA is prepared from the tissue sample. The nucleotide probe is preferably detectably labeled, such as with a fluorescent or radioactive label.

In one embodiment the nucleotide probe is a PCR primer that is used to amplify at least a portion of the MrgX2 RNA, if present, in the sample.

In a further aspect of the invention, a method of diagnosing melanoma in a patient comprises obtaining a tissue sample from the patient and isolating protein from the sample. The protein is then contacted with a probe that is specific for MrgX2, preferably MrgX2 comprising the amino acid sequence of SEQ ID NO: 4. The probe is preferably an antibody to MrgX2, more preferably a monoclonal antibody to MrgX2. The antibody is preferably labeled, such as with a fluorescent or radioactive label. In one embodiment the protein is separated according to size, for example on a polyacrylamide gel, prior to being contacted with the probe. In another embodiment the protein is immobilized on a membrane, such as a nitrocellulose membrane, prior to being contacted with the probe.

### BRIEF DESCRIPTION OF THE DRAWINGS

FIG. 1 is a Phylogenetic analysis of rat Mrgs and comparison to murine MrgBs. The two subgroups of mMrgB-related genes are shown.

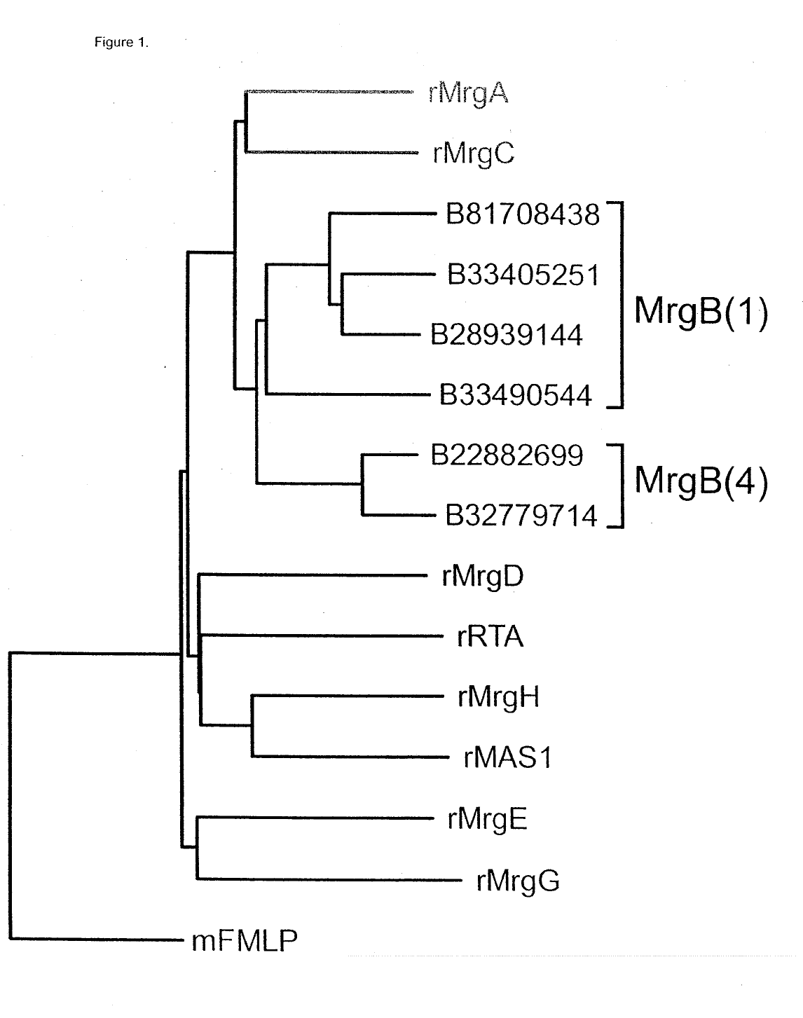

FIG. 2 shows in situ hybridization of adult mouse DRG sections with probes for mMrgB4 and mMrgB5. mMrgB4 and mMrgB5 are expressed in subsets of DRG sensory neurons. None of the other mouse MrgB genes are expressed in DRG neurons based on degenerate RT-PCR experiments.

FIG. 3 shows that murine MrgB1 is specifically expressed in skin and spleen at birth.

FIG. 4 illustrates expression of mMrgB1 in relation to melanocyte specific markers.

FIG. 5 illustrates that human MrgX2 is specifically expressed in melanoma cells.

### DETAILED DESCRIPTION

While it was initially reported that all members of the Mrg family of G protein coupled receptors are expressed in dorsal root ganglia (Dong et al., supra), it has been found that a subset of Mrgs is not expressed in dorsal root ganglia (DRG), but rather is expressed in a subset of cells in the skin. This subset of Mrg genes were found in mice, rats and humans and appears to represent a phylogenetically distinct subset of what were originally called the MrgB family of receptors. Further, it was discovered that at least one Mrg family member in humans, MrgX2, was exclusively expressed in melanoma tumor cells.

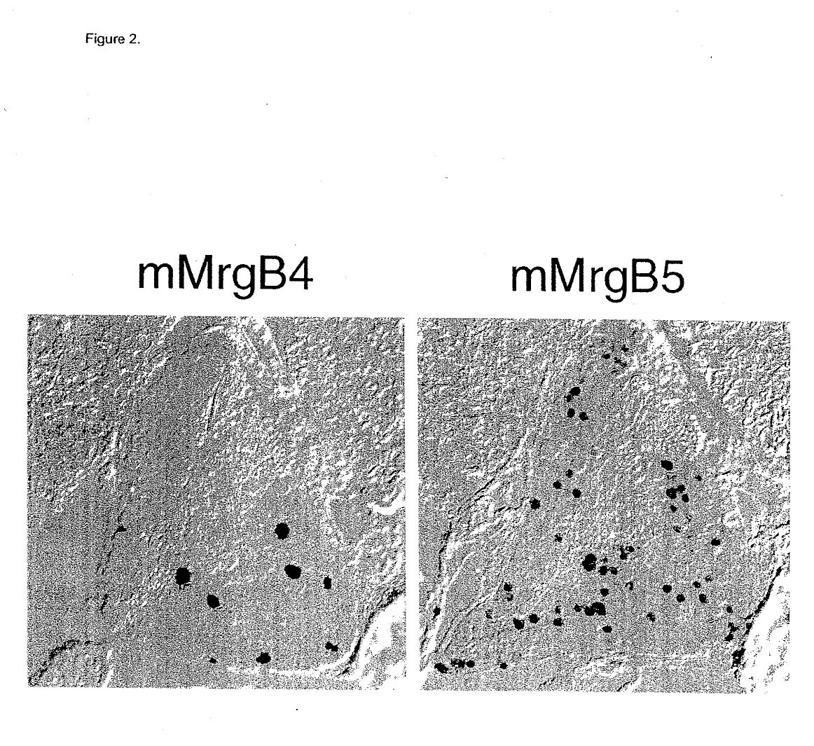

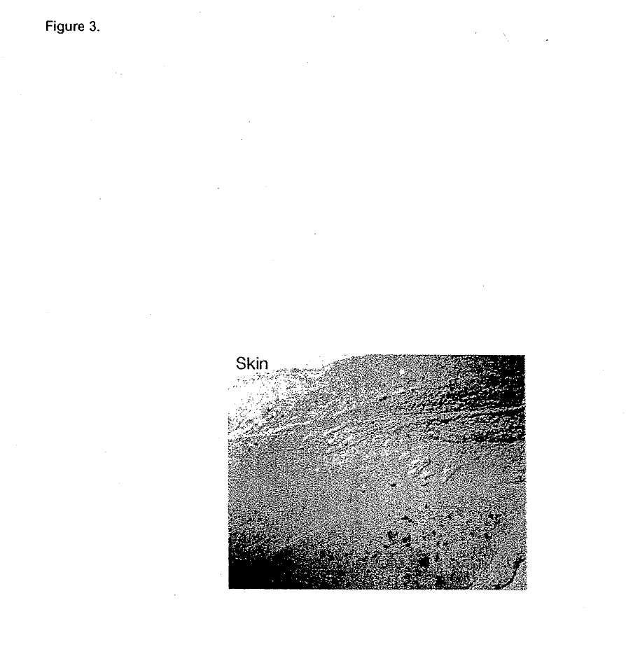

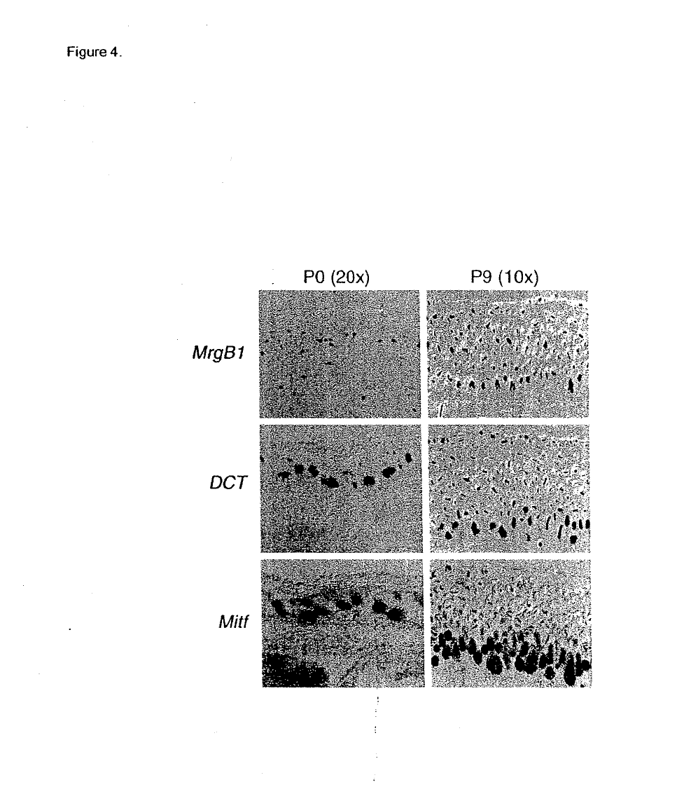

In the mouse, MrgB4 and MrgB5 were found to be specifically expressed in sensory neurons of adult dorsal root ganglia, like most other MrgB family members (Example 1, FIG. 2). In contrast, mMrgB1 was found to be specifically expressed in a subset of cells in the skin of postnatal mice, as well as in the spleen (Example 1, FIG. 3). No expression was detected elsewhere in the body. This distribution of mMrgB1 positive cells was found to be similar, but not identical, to the distribution of melanocytes in the skin at this time in development (Example 1, FIG. 4). In addition, the MrgB1 sequence was found to be equally as abundant as some specific melanocyte markers (e.g., DCT), in a cDNA library from B16-F10 murine melanoma cells, as shown in Table 1.

TABLE 1
Distribution of Clone Abundances in a cDNA
Library from Murine B16-F10 Melanoma Cells

| Gene | # Copies in B16-F10 EST Library | |
|---|---|---|
| TyRP1 | 292 | (1.6%) |
| Selenoprotein P | 192 | |
| Monoglyceride lipase | 174 | |
| Tyrosinase | 173 | |
| Sphyngomyelin phosphodiesterase 1 | 114 | |
| Melastatin | 107 | |
| MrgB1 | 96 | (0.5%) |
| Dopachrome tautomerase | 86 | |
| Endothelin B receptor | 75 | |
| Mitf | 8 | |
| p75, c-ret, Sox 10, MrgB2 | 0 | |

Notes:
17,989 EST sequences from the B16F10Y mouse melanoma cell line were assembled into transcript clusters using PHRAP. The nine most abundant transcripts are listed above. Five of the nine are well-characterized markers of the melanocyte lineage (TyRP-1, Tyrosinase, Melastatin, TyRP-2, and Endothelin-B receptor). The melanocyte lineage transcription factor Mitf is represented 8 times in this collection.

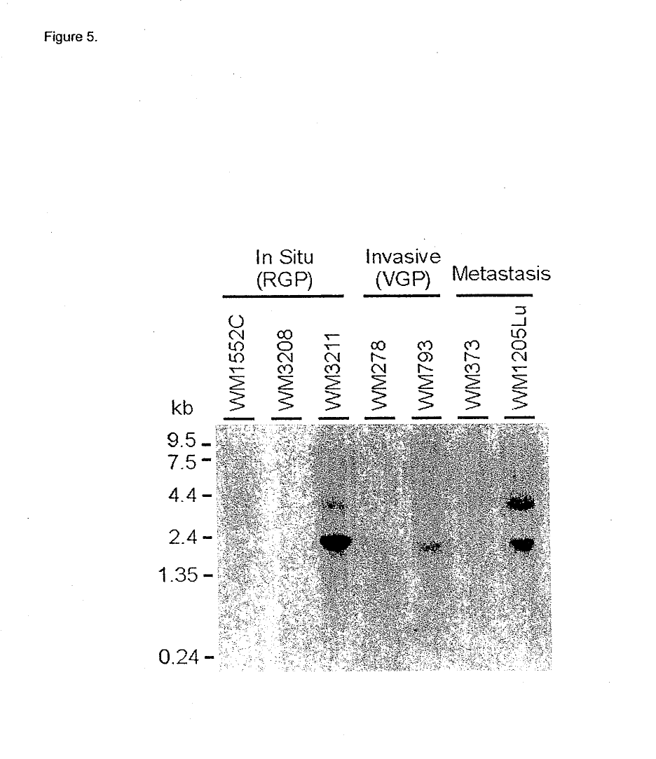

The expression of the four known human genes was also investigated. hMrgX1 was found to be expressed in human dorsal root ganglion, but hMrgX2 was not. However, hMrgX2 expression was observed in a number of human melanoma cell lines, several of which are derived from metastatic melanoma (Example 2, FIG. 5). Expression of hMrgX2 was not detected in any other tissue.

MrgB 1 and MrgX2 can serve as therapeutics and as a target for agents that modulate their expression or activity, for example in the treatment of melanoma. Importantly, antibodies to MrgB1 and MrgX2, as well as other binding proteins or compounds, may be used to identify melanoma in a patient.

### A. DEFINITIONS

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

When used herein the terms “Mrg,” “Mrg receptor” and “Mrg polypeptide” are used to refer to a group of Mrg receptors comprising MrgX2 and MrgB1, including native sequence molecules, variants and chimeric molecules.

As used herein, “MrgB1” and “MrgB1 polypeptide,” which are used interchangeably, refer to native sequence MrgB1 (SEQ ID NO: 2), MrgB1 variants, and chimeric MrgB1.

“MrgX2” and “MrgX2 polypeptide,” which are used interchangeably, refer to native sequence MrgX2 (SEQ ID NO: 4), MrgX2 variants, and chimeric MrgX2.

“Nucleic acid” is defined as RNA or DNA that encodes an Mrg polypeptide as defined above, or is complementary to a nucleic acid sequence encoding such polypeptide, or hybridizes to such RNA or DNA and remains stably bound to it under appropriate stringency conditions. Specifically included are genomic DNA, cDNA, mRNA and antisense molecules, as well as nucleic acids based on alternative backbones or including alternative bases whether derived from natural sources or synthesized.

As used herein, a nucleic acid molecule is said to be “isolated” when the nucleic acid molecule is substantially separated from contaminant nucleic acid molecules encoding other polypeptides.

“Stringent conditions” are those that (1) employ low ionic strength and high temperature for washing, for example, and without limitation, 0.015 M NaCl/0.0015 M sodium citrate/0.1% SDS at 50° C., or (2) employ during hybridization a denaturing agent such as formamide, for example, and without limitation, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

A “native” or “native sequence” Mrg polypeptide, such as MrgB1 or MrgX2, has the amino acid sequence of a naturally occurring Mrg receptor in any mammalian species (including humans), irrespective of its mode of preparation. Accordingly, a native or native sequence Mrg receptor may be isolated from nature, produced by techniques of recombinant DNA technology, chemically synthesized, or produced by any combinations of these or similar methods. Native Mrg receptors specifically include polypeptides having the amino acid sequence of naturally occurring truncated forms, allelic variants, isoforms or spliced variants of these receptors. Exemplary native sequence MrgX2 and MrgB1 amino acid sequences are provided in SEQ ID NOs: 4 and 2, respectively.

“Variants” are biologically active polypeptides having an amino acid sequence which differs from the sequence of a native sequence MrgB1 or MrgX2 polypeptide by virtue of an insertion, deletion, modification and/or substitution of one or more amino acid residues within the native sequence. Variants include peptide fragments of at least 5 amino acids, preferably at least 10 amino acids, more preferably at least 15 amino acids, even more preferably at least 20 amino acids that retain a biological activity of the corresponding native sequence polypeptide. Variants also include polypeptides wherein one or more amino acid residues are added at the N— or C-terminus of, or within, a native sequence. Further, variants also include polypeptides where a number of amino acid residues are deleted and optionally substituted by one or more different amino acid residues. Variants typically have less than 100% amino acid identity with a native sequence Mrg receptor, such as native sequence MrgB1 (SEQ ID NO: 2) or MrgX2 (SEQ ID NO: 4). However, a biologically active variant will preferably have at least about 60% amino acid identity with a naturally occurring Mrg, such as MrgB1 (SEQ ID NO: 2) or MrgX2 (SEQ ID NO: 4), more preferably at least about 70%, even more preferably about 80%, 85%, or 90%, with increasing preference to about 99%, in 1% increments.

As used herein, a “conservative variant” refers to a variant with one or more alterations in the amino acid sequence that do not adversely affect the biological functions of the protein. A substitution, insertion or deletion is said to adversely affect the protein when the altered sequence prevents or disrupts a biological fimction associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

Identity or homology with respect to amino acid sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

Proteins can be aligned, for example, using CLUSTALW (Thompson et al. Nucleic Acids Res 22:4673-80 (1994)) and homology or identity at the nucleotide or amino acid sequence level may be determined by BLAST (Basic Local Alignment Search Tool) analysis using the algorithm employed by the programs blastp, blastn, blastx, tblastn and tblastx (Karlin, et al. Proc. Natl. Acad. Sci. USA 87: 2264-2268 (1990) and Altschul, S. F. J. Mol. Evol. 36: 290-300 (1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

A “fragment” of an encoding nucleic acid molecule refers to a small portion of the entire protein coding sequence. The size of the fragment will be determined by the intended use. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming, as will be apparent to one of skill in the art.

“Functional derivatives” include amino acid sequence variants, and covalent derivatives of the native polypeptides as long as they retain a qualitative biological activity of the corresponding native polypeptide.

By “Mrg ligand” is meant a molecule which specifically binds to and preferably activates an Mrg receptor, particularly MrgB1 and/or MrgX2. Examples of Mrg ligands include, but are not limited to RF-amide neuropeptides, such as KiSS (Ohtaki et al. Nature 411:613-617 (2001)), FMRF, FLRF, NPAF, NPFF, and RFRP-1. The ability of a molecule to bind to an Mrg polypeptide can be determined, for example, by the ability of the putative ligand to bind to membrane fractions prepared from cells expressing the Mrg polypeptide.

A “chimeric” molecule is a polypeptide comprising a full-length polypeptide of the present invention, a variant, or fragment, such as one or more domains of an MrgB1 or MrgX2 polypeptide, fused or bonded to a heterologous polypeptide. The chimeric molecule will generally share at least one biological property in common with a naturally occurring native sequence polypeptide, or comprise an antigenic fragment of an Mrg polypeptide. An example of a chimeric molecule is one that is epitope tagged for purification purposes. Another chimeric molecule is an immunoadhesin.

The term “epitope-tagged” when used herein refers to a chimeric polypeptide comprising at least a portion of an Mrg polypeptide, such as MrgB1 or MrgX2, fused to a “tag polypeptide”. The tag polypeptide has enough residues to provide an epitope against which an antibody can be made, yet is short enough such that it does not interfere with the biological activity of the Mrg. The tag polypeptide preferably is fairly unique so that an antibody against it does not substantially cross-react with other epitopes. Suitable tag polypeptides generally have at least six amino acid residues and usually between about 8 and about 50 amino acid residues (preferably between about 9 and about 30 residues). Preferred are poly-histidine sequences, which bind nickel, allowing isolation of the tagged protein by Ni-NTA chromatography as described (See, e.g., Lindsay et al. Neuron 17:571-574 (1996)).

“Agonists” are molecules or compounds that stimulate one or more of the biological properties of MrgX2 and/or MrgB1. These may include, but are not limited to, small organic and inorganic molecules, peptides, peptide mimetics and agonist antibodies.

The term “antagonist” is used in the broadest sense and refers to any molecule or compound that blocks, inhibits or neutralizes, either partially or fully, a biological activity mediated by a receptor of the present invention by preventing the binding of an agonist. Antagonists may include, but are not limited to, small organic and inorganic molecules, peptides, peptide mimetics and neutralizing antibodies.

As used herein, a protein is said to be isolated when physical, mechanical or chemical methods are employed to remove the protein from cellular constituents that are normally associated with the protein. A skilled artisan can readily employ standard purification methods to obtain an isolated protein. In some instances, isolated proteins of the invention will have been separated or purified from many cellular constituents, but will still be associated with cellular membrane fragments or membrane constituents.

Thus, for example, “isolated MrgX2” means MrgX2 that has been purified from a protein source or has been prepared by recombinant or synthetic methods and purified. Purified Mrg is substantially free of other polypeptides or peptides. “Substantially free” here means less than about 5%, preferably less than about 2%, more preferably less than about 1%, even more preferably less than about 0.5%, most preferably less than about 0.1% contamination with other source proteins.

“Essentially pure” protein means a composition comprising at least about 90% by weight of the protein, based on total weight of the composition, preferably at least about 95% by weight, more preferably at least about 90% by weight, even more preferably at least about 95% by weight. “Essentially homogeneous” protein means a composition comprising at least about 99% by weight of protein, based on total weight of the composition.

“Biological property” is a biological or immunological activity, where biological activity refers to a biological function (either inhibitory or stimulatory) of a native sequence or variant polypeptide molecule herein. The ability to induce the production of an antibody against an epitope within such polypeptide, is referred to as immunological activity. Biological properties specifically include the ability to bind a naturally occurring ligand of the receptor molecules herein, preferably specific binding, and even more preferably specific binding with high affinity.

The term “mammal” is defined as an individual belonging to the class Mammalia and includes, without limitation, humans, domestic and farm animals, and zoo, sports, or pet animals, such as sheep, dogs, horses, cats or cows. Preferably, the mammal herein is human.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

“Native antibodies” and “native immunoglobulins” are usually heterotetrameric glycoproteins, composed of two identical light (L) chains and two identical heavy (H) chains. Each light chain is linked to a heavy chain by one covalent disulfide bond while The number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “antibody” is used in the broadest sense and specifically covers human, non-human (e.g. murine) and humanized monoclonal antibodies (including full length monoclonal antibodies), polyclonal antibodies, multi-specific antibodies (e.g., bispecific antibodies), and antibody fragments so long as they exhibit the desired specificity and/or activity.

“Antibody fragments” comprise a portion of a full-length antibody, generally the antigen binding or variable domain thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to an antibody obtained from a population of antibodies wherein the individual antibodies comprising the population are identical except for possible naturally occurring mutations that may be present in minor amounts. Monoclonal antibodies are highly specific and are directed against a single antigenic site. In addition, monoclonal antibodies may be made by any method known in the art. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. Fragments of chimeric antibodies are also included provided they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are antibodies that contain minimal sequence derived from non-human immunoglobulin. Humanized antibodies are generally human immunoglobulins in which hypervariable region residues are replaced by hypervariable region residues from a non-human species such as mouse, rat, rabbit or non-human primate having the desired specificity, affinity, and capacity. Framework region (FR) residues of the human immunoglobulin may be replaced by corresponding non-human residues. In addition, humanized antibodies may comprise residues that are not found in either the recipient antibody or in the donor antibody. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

The term “epitope” is used to refer to binding sites for (monoclonal or polyclonal) antibodies on protein antigens.

By “agonist antibody” is meant an antibody which is a ligand for an Mrg receptor, such as MrgX2 or MrgB1, and thus is able to activate and/or stimulate one or more of the effector functions of the native sequence receptor.

By “neutralizing antibody” is meant an antibody molecule which is able to block or significantly reduce an effector function of a polypeptide, particularly an Mrg polypeptide. For example, a neutralizing antibody may inhibit or reduce MrgX2 and/or MrgB1 activation by a known ligand.

The term “Mrg immunoadhesin” refers to a chimeric molecule that comprises at least a portion of an Mrg molecule (native or variant) and an immunoglobulin sequence. The immunoglobulin sequence preferably, but not necessarily, is an immunoglobulin constant domain. Immunoadhesins can possess many of the properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. If the two arms of the immunoadhesin structure have different specificities, the immunoadhesin is called a “bispecific immunoadhesin” by analogy to bispecific antibodies.

As used herein, “treatment” is a clinical intervention made in response to a disease, disorder or physiological condition manifested by a patient. The aim of treatment includes the alleviation or prevention of symptoms, slowing or stopping the progression or worsening of a disease, disorder, or condition and the remission of the disease, disorder or condition. “Treatment” refers to both therapeutic treatment and prophylactic or preventative measures. Those in need of treatment include those already affected by a disease or disorder or undesired physiological condition as well as those in which the disease or disorder or undesired physiological condition is to be prevented.

The term “effective amount” refers to an amount sufficient to effect beneficial or desirable clinical results. An effective amount of an agonist or antagonist is an amount that is effective to treat a disease, disorder or unwanted physiological condition.

The term “skin cancer” is used broadly to refer to the malignant proliferation of any cells of the skin. Melanoma is a form of skin cancer. In particular, “melanoma” refers to a malignant proliferation of melanocytes. The first phase of most melanomas is termed the radial growth phase (RGP) and is along the dermoepidermal junction and within the dermis. In the vertical growth phase (VGP) growth down through the epidermis brings the malignant melanocytes into contact with lymphatic tissue and capillaries, leading to metastasis. Melanomas are well known in the art and melanoma cell lines are widely available, for example from the Wistar Institute (USA).

“Pharmaceutically acceptable” carriers, excipients, or stabilizers are ones which are nontoxic to the cell or mammal being exposed thereto at the dosages and concentrations employed. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™.

B. MrgB1 and MrgX2

Mrg polypeptides that may be used in the present invention include, but are not limited to, native sequence molecules, fragments, variants and chimeric polypeptides. Polypeptide sequences of native MrgB1 and MrgX2 are provided in SEQ ID NOs: 2 and 4, respectively.

Contemplated variants include, for example, those that are naturally occurring and those that have been manipulated, such as by site-directed or PCR mutagenesis, as well as derivatives wherein the protein has been covalently modified by substitution, chemical, enzymatic, or other appropriate means with a moiety other than a naturally occurring amino acid (for example a detectable moiety such as an enzyme or radioisotope).

Variations in native sequence Mrg polypeptides, or in various domains identified therein, can be made using any techniques known in the art. Variation can be achieved, for example, by substitution of at least one amino acid with any other amino acid in one or more of the domains of the protein. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 or more amino acids.

Polypeptide fragments are also useful in the methods of the present invention. Such fragments may be truncated at the N-terminus or C-terminus, or may lack internal residues, for example, when compared with a fall-length native protein. Certain fragments lack amino acid residues that are not essential for a desired biological activity of the Mrg polypeptide.

Mrg fragments may be prepared by any of a number of conventional techniques. Desired peptide fragments may be chemically synthesized or generated by enzymatic digestion, such as by treating the protein with an enzyme known to cleave proteins at sites defined by particular amino acid residues. Alternatively, the DNA encoding the protein may be digested with suitable restriction enzymes and the desired fragment isolated. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, Mrg polypeptide fragments share at least one biological and/or immunological activity with a native Mrg polypeptide.

C. Nucleic Acid Molecules

Nucleic acid molecules that encode Mrg polypeptides, particularly MrgB1 and MrgX2 are used in various embodiments of the present invention. cDNA's encoding full length MrgB1 and MrgX2 are provided in SEQ ID NO: 1 and 3, respectively, and the corresponding deduced amino acid sequences are provided in SEQ ID NO: 2 and 4. The polynucleotides can be obtained by standard techniques well known to those of skill in the art, including hybridization screening and PCR.

Preferred molecules are those that hybridize under the above defined stringent conditions to the complement of a cDNA encoding an Mrg polypeptide, for example SEQ ID NO: 1 or SEQ ID NO: 3.

It is not intended that the methods of the present invention be limited by the source of the polynucleotide. The polynucleotide can be from a human or non-human mammal, derived from any recombinant source, synthesized in vitro or by chemical synthesis. The nucleotide may be DNA or RNA and may exist in a double-stranded, single-stranded or partially double-stranded form.

Nucleic acids useful in the present invention include, by way of example and not limitation, oligonucleotides such as antisense DNAs and/or RNAs; ribozymes; DNA for gene therapy; DNA and/or RNA chimeras; various structural forms of DNA including single-stranded DNA, double-stranded DNA, supercoiled DNA and/or triple-helix DNA; Z-DNA; mRNA, and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England).

Isolated or purified polynucleotides having at least 10 nucleotides (i.e., a hybridizable portion) of an Mrg polypeptide coding sequence or its complement are used in some embodiments. In other embodiments, the polynucleotides preferably comprise at least 25 (continuous) nucleotides, 50 nucleotides, 100 nucleotides, 150 nucleotides, or 200 nucleotides of an Mrg coding sequence, or a full-length Mrg coding sequence. Nucleic acids can be single or double stranded. Additionally, polynucleotides that selectively hybridize to a complement of the foregoing coding sequences are used in some embodiments.

Nucleotide sequences that encode a mutant of an Mrg protein, peptide fragments of Mrg, truncated forms of Mrg, and Mrg fusion proteins may also be useful in the methods of the present invention. Nucleotides encoding fusion proteins may include, but are not limited to, full length Mrg sequences, truncated forms of Mrg, or nucleotides encoding peptide fragments of Mrg fused to an unrelated protein or peptide, such as for example, a domain fused to an Ig Fc domain or fused to an enzyme such as a fluorescent protein or a luminescent protein which can be used as a marker.

Fragments of the nucleic acid molecules encoding Mrg polypeptides (i.e., synthetic oligonucleotides) may be used in some embodiments of the present invention, for example, as probes for the detection of Mrg polypeptides, or as specific primers for polymerase chain reaction (PCR). Such fragments can easily be synthesized by chemical techniques, for example, the phosphotriester method of Matteucci, et al., (J. Am. Chem. Soc. 103:3185-3191, 1981) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Any nucleotide sequence which encodes the amino acid sequence of an Mrg polypeptide can be used to generate recombinant molecules which direct the expression of the protein, as described in more detail below. In addition, the methods of the present invention may also utilize a fusion polynucleotide comprising an Mrg coding sequence and a second coding sequence for a heterologous protein.

D. Recombinant DNA Molecules Containing a Nucleic Acid Molecule

Recombinant DNA molecules (rDNAs) that contain an Mrg polypeptide coding sequence are also useful in the present invention. As used herein, a rDNA molecule is a DNA molecule that has been subjected to molecular manipulation in situ. Methods for generating rDNA molecules are well known in the art, for example, see Sambrook et al., Molecular Cloning: A Laboratory Manual, 2nd edition, 1989; Cold Spring Harbor Laboratory Press, Cold Spring Harbor, N.Y. In the preferred rDNA molecules, a coding DNA sequence is operably linked to expression control sequences and/or vector sequences.

Thus the present invention also contemplates DNA vectors that contain an Mrg coding sequence and/or its complement, optionally associated with a regulatory element that directs the expression of the coding sequences. The choice of vector and/or expression control sequences to which the encoding sequence is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule. Such vectors are well known in the art and include, but are not limited to, pUC8, pUC9, pBR322 and pBR329 available from BioRad Laboratories, (Richmond, Calif.), pPL and pKK223 available from Pharmacia (Piscataway, N.J.) for use in prokaryotic cells, and pSVL and pKSV-10 (Pharmacia), pBPV-1/pML2d (International Biotechnologies, Inc.), pCDNA and pTDT1 (ATCC, #31255), for use in eukaryotic cells, as well as eukaryotic viral vectors such as adenoviral or retroviral vectors. In addition, vectors may include a selection gene whose expression confers a detectable marker such as a drug resistance. Typical selection genes encode proteins that confer resistance to antibiotics or other toxins, e.g., ampicillin, neomycin, methotrexate, or tetracycline, complement auxotrophic deficiencies, or supply critical nutrients withheld from the media. Such selection systems are well known in the art The selectable marker can optionally be present on a separate plasmid and introduced by co-transfection.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment Chinese hamster ovary (CHO) cells deficient in DHFR activity are prepared and propagated as described by Urlaub et al., Proc. Natl. Acad. Sci. USA, 77:4216 (1980). The CHO cells are then transformed with the DHFR selection gene and transformants are are identified by culturing in a culture medium that contains methotrexate (Mtx), a competitive antagonist of DHFR. The transformed cells are then exposed to increased levels of methotrexate. This leads to the synthesis of multiple copies of the DHFR gene, and, concomitantly, multiple copies of other DNA comprising the expression vectors, such as the DNA encoding the protein of interest, for example DNA encoding an Mrg polypeptide.

Other methods, vectors, and host cells suitable for adaptation to the synthesis of an Mrg polypeptide in recombinant vertebrate cell culture are well known in the art and are readily adapted to the specific circumstances.

E. Host Cells Containing an Exogenously Supplied Coding Nucleic Acid Molecule

Host cells transformed with a nucleic acid molecule that encodes an Mrg polypeptide are also provided. The host cell can be either prokaryotic or eukaryotic but is preferably eukaryotic.

Eukaryotic cells useful for expression of a protein of the invention are not limited, so long as the cell line is compatible with cell culture methods and compatible with the propagation of the expression vector and expression of the gene product. Such host cells are capable of complex processing and glycosylation activities. In principle, any higher eukaryotic cell culture is workable, whether from vertebrate or invertebrate culture. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), HEK293 cells and other known eukaryotic tissue culture cell lines. Additional examples of useful mammalian host cell lines that can be readily cultured are monkey kidney CV1 line transformed by SV40 (COS-7, ATCC CRL 1651); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human liver cells (Hep G2, HB 8065); and mouse mammary tumor (MMT 060562, ATCC CCL51).

Melanoma cell lines are preferably used in some embodiments. Exemplary melanoma cell lines include, for example, SK-MEL cell lines, IST-MEL cell lines, IGR cell lines, COLO cell lines, cell lines, and cell lines derived from B16. Human Wistar Melanoma (WM) cell lines are preferably used in some embodiments (See The Wistar Melanoma (WM) Cell Lines in HUMAN CELL CULTURE, Vol. I, 259-274, 1999). Preferred WM cell lines include WM1205Lu and WM793. In other embodiments melanoma cell lines derived from B16, such as B16-F10 are used (See, e.g., Riley Ann. N.Y. Acad. Sci. 100:762-790 (1963); Silagi J. Cell Biol. 43:263-274 (1969)).

Propagation of vertebrate cells in culture is a routine procedure. See, e.g., Tissue Culture, Academic Press, Kruse and Patterson, editors (1973).

Any prokaryotic host can be used to express a rDNA molecule encoding a protein or a protein fragment of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. Proc. Natl. Acad. Sci. USA 69:2110, (1972); and Maniatis et al., Molecular Cloning, A Laboratory Manual, Cold Spring Harbor Laboratory, Cold Spring Harbor, N.Y. (1982). With regard to transformation of vertebrate cells with vectors containing rDNAs, electroporation, cationic lipid or salt treatment methods are typically employed, see, for example, Graham et al. Virol. 52:456, (1973); Wigler et al. Proc. Natl. Acad. Sci. USA 76:1373-76, (1979). The calcium phosphate precipitation method is preferred. However, other methods of for introducing DNA into cells may also be used, including nuclear microinjection and bacterial protoplast fusion.

For transient expression of Mrg receptors and measurement of intracellular Ca²⁺ changes in response to receptor activation, HEK cells can be co-transfected with Mrg expression constructs and a fluorescent reporter gene using the calcium-phosphate precipitation technique (see, e.g. Han et al. Proc. Natl. Acad. Sci. USA 99:14740-14745 (2002)). HEK293 cells are typically grown in high glucose DMEM (Life Technologies) supplemented with 10% fetal calf serum (Life Technologies).

Prokaryotic cells used to produce Mrg polypeptides are cultured in suitable media as described generally in Sambrook et al, supra.

The host cells referred to in this disclosure encompass cells in culture as well as cells that are within a host animal.

Gene amplification and/or expression may be measured by any technique known in the art, including Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Various labels may be employed, most commonly radioisotopes, particularly ³²P. Immunological methods for measuring gene expression include immunohistochemical staining of tissue sections or cells in culture, as well as assaying protein levels in culture medium or body fluids. With immunohistochemical staining techniques, a cell sample is prepared by dehydration and fixation, followed by reaction with labeled antibodies specific for the gene product, where the labels are usually visually detectable, such as enzymatic labels, fluorescent labels, luminescent labels, and the like.

Antibodies useful for immunohistochemical staining and/or assay of a sample may be either monoclonal or polyclonal, and may be prepared as described herein.

F. Modifications of Mrg Polypeptides

Covalent modifications of Mrg polypeptides and their respective variants may be made, as may modifications of antibodies to the Mrg polypeptides. In one embodiment, specific amino acid residues of a polypeptide are reacted with an organic derivatizing agent. Derivatization with bifunctional agents is useful, for instance, for crosslinking Mrg or Mrg fragments or derivatives to a water-insoluble support matrix or surface for use in methods for purifying anti-Mrg antibodies and identifying binding partners and ligands. In addition, Mrg or Mrg fragments may be crosslinked to each other to modulate binding specificity and effector function. Many crosslinking agents are known in the art and include, but are not limited to, 1,1-bis(diazoacetyl)-2-phenylethane, glutaraldehyde, N-hydroxysuccinimide esters, bifunctional maleimides such as bis-N-maleimido-1,8-octane and agents such as methyl-3-[(p-azidophenyl)dithio]propioimidate.

Other contemplated modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Modification of the glycosylation patterns of the polypeptides are also contemplated. Methods for altering the glycosylation pattern of polypeptides are well known in the art. For example, one or more of the carbohydrate moities found in native sequence Mrg may be removed chemically, enzymatically or by modifying the glycosylation site. Alternatively, additional gycosylation can be added, such as by manipulating the composition of the carbohydrate moities directly or by adding glycosylation sites not present in the native sequence Mrg by altering the amino acid sequence.

Another type of covalent modification of the polypeptides of the invention comprises linking the polypeptide or a fragment or derivative thereof to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The polypeptides of the present invention may also be modified in a way to form a chimeric molecule comprising an Mrg polypeptide fused to another, heterologous polypeptide or amino acid sequence.

In one embodiment, such a chimeric molecule comprises a fusion of the Mrg polypeptide with a tag polypeptide that provides an epitope to which an anti-tag antibody can selectively bind. The epitope tag is generally placed at the amino- or carboxyl-terminus of the polypeptide. The epitope tag allows for identification of the chimeric protein as well as purification of the chimeric protein by affinity purification using an anti-tag antibody or another type of affinity matrix that binds to the epitope tag. A number of tag polypeptides and their respective antibodies are well known in the art. Well known tags include poly-histidine (poly-his) or poly-histidine-glycine (poly-his-gly) tags; the flue HA tag polypeptide (Field et al., Mol. Cell. Biol., 8:2159-2165 (1988)); the c-myc tag (Evan et al., Molecular and Cellular Biology, 5:3610-3616 (1985)); the Herpes Simplex virus glycoprotein D (gD) tag (Paborsky et al., Protein Engineering, 3(6):547-553 (1990)) and the Flag-peptide (Hopp et al., BioTechnology, 6:1204-1210 (1988)).

In another embodiment, the chimeric molecule comprises a fusion of Mrg with an immunoglobulin or a particular region of an immunoglobulin. To produce an immunoadhesin, the polypeptide of the invention or a fragment or specific domain(s) thereof could be fused to the Fc region of an IgG molecule. Typically the fusion is to an immunoglobulin heavy chain constant region sequence. Mrg-immunoglobulin chimeras for use in the present invention are normally prepared from nucleic acid encoding one or more extracellular domains, or fragments thereof, of an Mrg receptor fused C-terminally to nucleic acid encoding the N-terminus of an immunoglobulin constant domain sequence. N-terminal fusions are also possible.

While not required in the immunoadhesins of the present invention, an immunoglobulin light chain might be present either covalently linked to an Mrg-immunoglobulin heavy chain fusion polypeptide, or directly fused to Mrg. In order to obtain covalent association, DNA encoding an immunoglobulin light chain may be coexpressed with the DNA encoding the Mrg-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs.

In yet another embodiment, the chimeric molecule of the present invention comprises a fusion of Mrg or a fragment or domain(s) thereof, with a heterologous receptor or fragment or domain(s) thereof. The heterologous receptor may be a related Mrg family member, or may be completely unrelated. The heterologous protein fused to the Mrg protein may be chosen to obtain a fusion protein with a desired ligand specificity or a desired affinity for a particular ligand or to obtain a fusion protein with a desired effector function.

G. Antibodies to Mrg Polypeptides

Antibodies are preferably prepared by standard methods well-known in the art. The subject antibody compositions may be polyclonal, such that a heterogeneous population of antibodies differing by specificity is present, or monoclonal, in which a homogeneous population of identical antibodies that have the same specificity for an Mrg polypeptide are present. While both monoclonal and polyclonal antibodies may be used in the methods of the subject invention, in many preferred embodiments, the subject antibodies are monoclonal antibodies.

Generally, an antigen or immunogen that can elicit an immune response characterized by the presence of antibodies of the subject invention is employed. The immunogen preferably comprises at least a portion of an Mrg polypeptide. For example, the immunogen may be a portion of a native sequence MrgB1 polypeptide (SEQ ID NO: 2) or a portion of a native sequence MrgX2 polypeptide (SEQ ID NO: 4).

Although methods of making monoclonal and polyclonal antibodies are well known in the art, preferred methods are briefly described herein. Variations of the following methods will be apparent to one of skill in the art.

For preparation of polyclonal antibodies, the first step is immunization of the host animal with the immunogen. To increase the immune response of the host animal, the immunogen may be combined with an adjuvant. Suitable adjuvants include alum, dextran, sulfate, large polymeric anions, oil & water emulsions, e.g. Freund's adjuvant, Freund's complete adjuvant, and the like. The immunogen may also be conjugated to synthetic carrier proteins or synthetic antigens. A variety of hosts may be immunized to produce the polyclonal antibodies. Such hosts include without limitation, rabbits, guinea pigs, other rodents such as mice or rats, sheep, goats, primates and the like. The immunogen is administered to the host, usually intradermally, with an initial dosage followed by one or more, usually at least two, additional booster dosages. Following immunization, the blood from the host is collected, followed by separation of the serum from the blood cells. The Ig present in the resultant antiserum may be further fractionated using known methods, such as ammonium salt fractionation, DEAE chromatography, and the like.

As with the preparation of polyclonal antibodies, the first step in preparing monoclonal antibodies specific for an epitope within the huntingtin protein, is to immunize a suitable host. Suitable hosts include rats, hamsters, mice, monkeys and the like, and are preferably mice. Monoclonal antibodies may be generated using the hybridoma method described by Kohler et al., Nature, 256:495 (1975) or by recombinant DNA methods, such as those described in U.S. Pat. No. 4,816,567.

The immunogen is administered to the host in any convenient manner known in the art. For example, and without limitation, administration may be by subcutaneous injection with adjuvants, nitrocellulose implants comprising the immunogen or intrasplenic injections. Alternatively, lymphocytes may be immunized in vitro. The immunization protocol may be modulated to obtain a desired type of antibody, e.g. IgG or IgM, where such methods are known in the art (Kohler and Milstein, Nature, 256:495 (1975)). Booster immunizations may be made, for example one month after the initial immunization. Animals are bled and analyzed for antibody titer. Boosting may be continued until antibody production plateaus.

Following immunization, plasma cells are harvested from the immunized host. Sources of plasma cells include the spleen and lymph nodes, with the spleen being preferred.

The plasma cells are then immortalized by fusion with myeloma cells to produce hybridoma cells. Fusion may be carried out by an electrocell fusion process or by using a suitable fusing agent, such as polyethylene glycol, to form a hybridoma cell (Goding, Monoclonal Antibodies: Principles and Practice, pp. 59-109, [Academic Press, 1996]). The plasma and myeloma cells are typically fused by combining the cells in a fusion medium usually in a ratio of about 10 plasma cells to 1 myeloma cell, where suitable fusion mediums include a fusion agent, e.g. PEG 1000, and the like. Following fusion, the fused cells will be selected, e.g. by growing on HAT medium.

A variety of myeloma cell lines are available. Preferably, the myeloma cell is HGPRT negative, incapable of producing or secreting its own antibodies, and growth stable. Preferred myeloma cells also fuse efficiently and support stable high-level production of antibody by the selected antibody-producing cells. Among these, preferred myeloma cell lines are murine myeloma lines, such as those derived from MOP-21 and MC.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133: 3001 (1984); Brodeur et al., Monoclonal Antibody Production Techniques and Applications, pp. 51-63, Marcel Dekker, Inc., New York, [1987]). Specific cell lines of interest include, for example, p3U1, SP 2/0 Ag14 and P3.x.63Ag8.653.

Following hybridoma cell production, culture supernatant from individual hybridomas is screened for reactivity with Mrg polypeptide. Such screening techniques are well known in the art and include radioimmunoassay (RIA), enzyme-linked immunosorent assay (ELISA), dot blot immunoassays, Western blots and the like. The binding affinity of the monoclonal antibody may, for example, be determined by the Scatchard analysis (Munson et al., Anal. Biochem., 107:220 (1980)).

After hybridoma cells secreting antibodies with the desired specificity, affinity and/or activity are selected, the cells may be subcloned by limiting dilution procedures and grown by standard methods (Goding, Monoclonal Antibodies: Principles and Practice, pp. 59-103, Academic Press, 1996). Culture media may be for example DMEM or RPMI-1640 medium. Alternatively, hybridomas may be grown in vitro as ascites tumors in an animal.

The desired antibody may be purified from the supernatants or ascites fluid by conventional techniques, e.g. affinity chromatography using Mrg polypeptide bound to an insoluble support, such as protein A sepharose.

DNA encoding the monoclonal antibody may be isolated and sequenced using conventional procedures, with the hybridoma cells serving as a source of the DNA. The isolated DNA may be introduced into host cells in culture to synthesize the monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. 81, 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-Huntingtin protein described herein.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pp. 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Mendez et al. (Nature Genetics 15: 146-156 [1997]) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Alternatively, phage display technology (McCafferty et al., Nature 348, 552-553 [1990]) can be used to produce human antibodies and antibody fragments in vitro, from immunoglobulin variable (V) domain gene repertoires from unimmunized donors.

Binding fragments or binding mimetics of the Mrg polypeptide antibodies may also be prepared. These fragments and mimetics preferably share the binding characteristics of one or more monoclonal antibodies. “Binding characteristics” when used herein include specificity, affinity, avidity, etc. for an Mrg polypeptide.

Antibody fragments, such as Fv and Fab may be prepared by cleavage of the intact protein, e.g. by protease or chemical cleavage. Nucleic acid encoding the antibody fragments or binding mimetics may be identified.

Antibody fragments, such as single chain antibodies or scFvs, may also be produced by recombinant DNA technology where such recombinant antibody fragments retain the binding characteristics of the above antibodies. “Antibody fragments” when used herein refer to a portion of an intact antibody, such as the antigen binding or variable region and may include single-chain antibodies, Fab, Fab′, F(ab′)2 and Fv fragments, diabodies, linear antibodies, and multispecific antibodies generated from portions of intact antibodies.

Recombinantly produced antibody fragments generally include at least the V_{H} and V_{L} domains of the subject antibodies, so as to retain the desired binding characteristics. These recombinantly produced antibody fragments or mimetics may be readily prepared from the antibodies of the present invention using any convenient methodology, such as the methodology disclosed in U.S. Pat. Nos. 5,851,829 and 5,965,371; the disclosures of which are herein incorporated by reference. The antibody fragments or mimetics may also be readily isolated from a human scFvs phage library (Pini et al., Curr. Protein Pept. Sci., 1(2):155-69 (2000)) using Mrg polypeptide.

The subject antibodies are modified to optimize their utility, for example for use in a particular immunoassay or their therapeutic use.

In one embodiment the antibodies are modified by the attachment of a moiety that allows for their visualization upon binding to an Mrg polypeptide, as described in more detail below. For example, the antibodies may be modified with a radioactive moiety, or with an fluorescent tag. Such modifications are well known in the art.

In another embodiment the antibodies are conjugated with a compound to increase their therapeutic utility. For example, the antibodies may be modified with a toxic compound that kills cells expressing the appropriate antigen, such as an Mrg polypeptide. Particularly preferred are small molecule toxins. Exemplary toxins include ricins, such as ricin A, diptheria toxins, maytanisinoids, pseudomonas exotoxin, and radionuclides. Other toxins that can be conjugated to the anti-Mrg antibodies will be apparent to one of skill in the art.

I. Diagnostic Applications

The use of molecular biological tools has become routine in medicine. In one embodiment of the present invention, nucleic acid probes are used to determine the expression of a nucleic acid molecule comprising all or at least part of an Mrg encoding sequence in pathology specimens, as described below. In other embodiments antibodies are used to detect expression of an Mrg polypeptide. As MrgX2 was found to be specifically expressed in skin cancer cells, particularly melanoma cells, the detection of MrgX2 in a sample from a patient serves as an indication that a patient is suffering from skin cancer, such as melanoma.

Assays to detect nucleic acid or protein molecules of the invention may be in any available format. Typical assays for detecting Mrg encoding nucleic acid molecules include hybridization or PCR based formats. Typical assays for the detection of proteins, polypeptides or peptides of the invention include the use of antibody probes in any available format such as in situ binding assays, dot blots, western blots etc. See Harlow et al., Antibodies: A Laboratory Manual, Cold Spring Harbor Laboratory, 1988 and Section G. In preferred embodiments, assays are carried-out with appropriate controls.

In a typical assay, a sample of cells is obtained from a patient, such as a human subject. The patient may be suspected of suffering from melanoma or may be at an elevated risk of developing the disease. The sample is generally a physiological sample from the patient such as blood or tissue, preferably skin. Depending on the nature of the sample, it may or may not be pretreated prior to assay, as will be apparent to one of skill in the art. For example, in one embodiment the tissues are treated with collagenases or other proteases to make them amenable to cell lysis (Semenov et al, Biull Eksp Biol Med 104(7): 113-6 (1987)).

In one embodiment, mRNA expression may be monitored directly by hybridization to nucleic acid probes. Total RNA or mRNA is isolated from a tissue sample by standard procedures such those disclosed in Sambrook et al. (Molecular Cloning: A Laboratory Manual, 2nd Ed. Cold Spring Harbor Laboratory Press, 1989).

Probes to detect RNA expression level are preferably prepared from nucleic acids encoding at least a portion of an Mrg polypeptide, preferably MrgX2. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed using methods known in the art. For instance, the G+C content of the probe and the probe length can affect probe binding to its target sequence. Methods to optimize probe specificity are commonly available in Sambrook et al. (Molecular Cloning: A Laboratory Manual, 2nd Ed. Cold Spring Harbor Laboratory Press, NY, 1989) or Ausubel et al. (Current Protocols in Molecular Biology, Greene Publishing Co., NY, 1995).

Hybridization conditions are modified using known methods, such as those described by Sambrook et al. and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or porous glass wafer. The wafer can then be exposed to total cellular RNA or polyA RNA from a sample under conditions in which the affixed sequences will specifically hybridize. Such wafers and hybridization methods are widely available, for example, those disclosed by Beattie (WO 95/11755). By examining for the ability of a given probe to specifically hybridize to an RNA sample prepared from a sample obtained by a patient, expression of an Mrg polypeptide can be determined. Expression of MrgX2 indicates that the sample comprises melanoma tumor cells.

In other embodiments PCR is used to amplify Mrg nucleic acid sequences. Such assays are well known in the art (See, for example, PCR Protocols: A Guide to Methods and Applications ed. Inris, M., Gelfand, D., Sninsky, J. and White, T. Academic Press, San Diego (1990)).

A number of different immunoassay formats are known in the art and may be employed in detecting the presence of an Mrg polypeptide in a sample. Thus, in another embodiment antibodies that are specific for MrgX2 are used in immunoassays that are capable of providing for the detection of MrgX2 in a sample.

Immunoassays that may be used include but are not limited to Western blots on protein gels or protein spots on filters, where the antibody is labeled. Such assays are well known in the art. A variety of protein labeling schemes are known in the art and may be employed, the particular scheme and label chosen being the one most convenient for the intended use of the antibody, e.g. immunoassay. Examples of labels include labels that permit both the direct and indirect measurement of the presence of the antibody. Examples of labels that permit direct measurement of the antibody include radiolabels, such as ³H or ¹²⁵I, fluorescent dyes, beads, chemilumninescers and colloidal particles.

Examples of labels which permit indirect measurement of the presence of the antibody include enzymes where a substrate may provided for a colored or fluorescent product. For example, the antibodies may be labeled with a covalently bound enzyme capable of providing a detectable product signal after addition of suitable substrate. Instead of covalently binding the enzyme to the antibody, the antibody may be modified to comprise a first member of a specific binding pair which specifically binds with a second member of the specific binding pair that in conjugated to the enzyme, e.g. the antibody may be covalently bound to biotin and the enzyme conjugate to streptavidin. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

Other immunoassays include those based on a competitive formats, as are known in the art. One such format would be where a solid support is coated with the Mrg polypeptide. Labeled antibody is then combined with a sample suspected of having the protein of interest to produce a reaction mixture which, following sufficient incubation time for binding complexes to form, is contacted with the solid phase bound protein. The amount of labeled antibody which binds to the solid phase will be proportional to the amount of protein in the sample, and the presence of protein may therefore be detected. Other competitive formats that may be employed include those where the sample suspected of comprising protein is combined with a known amount of labeled protein and then contacted with a solid support coated with antibody specific for the protein. Such assay formats are known in the art and further described in Antibodies, A Laboratory Manual (Cold Springs Harbor Press (Cold Springs Harbor, N.Y. 1989)).

In immunoassays involving solid supports, the solid support may be any compositions to which antibodies or fragments thereof can be bound, which is readily separated from soluble material, and which is otherwise compatible with the overall immunoassay method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Before adding patient samples or fractions thereof, the non-specific binding sites on the insoluble support i.e. those not occupied by the first antibody, are generally blocked. Preferred blocking agents include non-interfering proteins such as bovine serum albumin, casein, gelatin, and the like. Alternatively, detergents, such as Tween, NP40 or TX100 may be used at non-interfering concentrations.

It is particularly convenient in a clinical setting to perform the immunoassay in a self-contained apparatus, and such devices are provided by the subject invention. A number of such devices and methods for their use are known in the art. The apparatus will generally employ a continuous flow-path over a suitable filter or membrane, and will have at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it a first antibody. The second, labeled antibody combined with the assayed sample is introduced and the sandwich assay performed as above.

In a particular embodiment a sample of cells from a patient are incubated with a labeled anti-MrgX2 antibody. After allowing time for binding, unbound antibody is removed. Any specific binding of the antibody to the cells is then visualized. Specific antibody binding indicates that the sample included malignant melanoma cells.

In another embodiment protein is prepared from a sample of cells from a patient and run on a polyacrylamide gel. The gel is then incubated with anti-MrgX2 antibody and binding is detected. Such methods are well known in the art. Specific binding of the anti-MrgX2 antibody indicates that the sample comprised the MrgX2 receptor and therefore that the patient suffered from melanoma.

K. Methods to Identify Agents that Modulate at Least One Mrg Activity

Several peptides have been putatively identified as endogenous ligands for Mrg receptors, in particular the RF-amide peptides, including KiSS, NPAF and NPFF. Thus, another embodiment of the present invention provides methods of isolating and identifying ligands of proteins of the invention that can be used in the treatment of melanoma.

1. Identification of Agonists and Antagonists

The present invention provides for assays to identify Mrg agonists or antagonists. Agents that are able to modulate the activity of an Mrg polypeptide are then tested for their utility in the treatment of melanoma, as described below. The assays for identifying compounds that can modulate Mrg polypeptide activity may be done in vitro or in vivo, by monitoring the response of a cell following binding of an Mrg ligand to the receptor. An Mrg agonist will produce a cellular response, while an antagonist will have no effect on cellular response but will be capable of preventing cellular response to a known agonist.

A variety of different types of agents can be assayed for agonistic or antagonistic activity, including without limitation, peptides, small molecules, nucleic acids and proteins.

Small molecules may have the ability to act as Mrg agonists or antagonists and thus may be screened for an effect on a biological activity of Mrg. Small molecules preferably have a molecular weight of less than 10 kD, more preferably less than 5 kD and even more preferably less than 2 kD. Such small molecules may include naturally occurring small molecules, synthetic organic or inorganic compounds, peptides and peptide mimetics. However, small molecules in the present invention are not limited to these forms. Extensive libraries of small molecules are commercially available and a wide variety of assays are well known in the art to screen these molecules for the desired activity.

Candidate Mrg agonist and antagonist small molecules are preferably first identified in an assay that allows for the rapid identification of potential agonists and antagonists. An example of such an assay is a binding assay wherein the ability of the candidate molecule to bind to the Mrg receptor is measured. In another example, the ability of candidate molecules to interfere with the binding of a known ligand, for example an RFamide peptide, is measured. Candidate molecules that are identified by their ability to bind to Mrg proteins or interfere with the binding of known ligands are then tested for their ability to stimulate one or more biological activities.

The activity of the Mrg polypeptides may be monitored in cells in which they are expressed by assaying for physiological changes in the cells upon exposure to the agent or agents to be tested. Such physiological changes include but are not limited to the flow of current across the membrane of the cell and changes in intracellular calcium concentrations.

In one embodiment an Mrg polypeptide, preferably MrgX2, is expressed in a cell that is capable of producing a second messenger response and that does not normally express an Mrg polypeptide. The cell is then contacted with the compound of interest and changes in the second messenger response are measured. Methods to monitor or assay these changes are readily available. For instance, the Mrg polypeptide may be expressed in cells expressing Gal5, a G protein a subunit that links receptor activation to increases in intracellular calcium [Ca²+] which can be monitored at the single cell level using the FURA-2 calcium indicator dye as disclosed in Chandrashekar et al. Cell 100:703-711, (2000). This assay is described in more detail in Han et al., supra.

In a further embodiment, agonists are identified or confirmed in cells that express endogenous Mrg polypeptides. Human or murine melanoma cell lines that naturally express MrgX2 or MrgB1, such as the B16-F10 cell line, are exposed to a compound of interest, such as a potential agonist. Changes in second messenger activity are then measured, such as by measuring a change in intracellular calcium concentrations.

The action of an agonist through an Mrg polypeptide can be confirmed by determining whether the observed activity is blocked by blocking the activity of the Mrg polypeptide, such as with a known antagonist or blocking antibody, or by interfering with expression of the Mrg polypeptide through the use of antisense or RNA interference.

Similar assays may also be used to identify inhibitors or antagonists of Mrg activation. For example, cells expressing Mrg and capable of producing a quantifiable response to receptor activation are contacted with a known Mrg activator and the compound to be tested. In one embodiment, HEK cells expressing Gα15 and an Mrg polypeptide are contacted with an RFamide peptide and the compound to be tested. The cellular response is measured, in this case an increase in [Ca²⁺]. A decreased response compared to the known activator by itself indicates that the compound acts as an inhibitor of activation.

While such assays may be formatted in any manner, particularly preferred formats are those that allow high-throughput screening (HTP). In HTP assays of the invention, it is possible to screen thousands of different modulators or ligands in a single day. For instance, each well of a microtiter plate can be used to run a separate assay, for instance an assay based on the ability of the test compounds to modulate receptor activation derived increases in intracellular calcium as described above.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

Antibodies that are immunoreactive with critical positions of an Mrg polypeptide, particularly MrgX2, can also be assayed for agonistic or antagonistic (neutralizing) activity. These antibodies may be human or non-human, polyclonal or monoclonal. They include amino acid sequence variants, glycosylation variants and fragments of antibodies. Antibody agents are obtained by immunization of suitable mammalian subjects with peptides, containing as antigenic regions, those portions of the protein intended to be targeted by the antibodies. General techniques for the production of such antibodies and the selection of agonist or neutralizing antibodies are well known in the art.

Mrg agonist and neutralizing antibodies may be preliminarily identified based on their ability to bind the Mrg receptor. For example, Western blot techniques well known in the art may be used to screen a variety of antibodies for their ability to bind Mrg. Mrg agonist and neutralizing antibodies are then identified from the group of candidate antibodies based on their biological activity. In one embodiment, Mrg agonist antibodies are identified by their ability to induce activation of a second messenger system in cells expressing the Mrg protein and comprising a second messenger system. For example, HEK cells overexpressing Gα15 and transfected with mrg may be contacted with a potential Mrg agonist antibody. An increase in intracellular calcium, measured as described in Example 5, would indicate that the antibody is an agonist antibody.

Identification of a neutralizing antibody involves contacting a cell expressing Mrg with a known Mrg ligand, such as an RFamide peptide, and the candidate antibody and observing the effect of the antibody on Mrg activation. In one embodiment, Mrg receptors expressed in HEK cells overexpressing Gα15 are contacted with an Mrg ligand such as FMRFamide and the candidate neutralizing antibody. A decrease in responsiveness to the ligand would indicate that the antibody is a neutralizing antibody.

The Mrg antagonists are not limited to Mrg ligands. Other antagonists include variants of a native Mrg receptor that retains the ability to bind an endogenous ligand but is not able to mediate a biological response. Soluble receptors and immunoadhesins that bind Mrg or drg-12 ligands may also be antagonists, as may antibodies that specifically bind a ligand near its binding site and prevent its interaction with the native receptor. These antagonists may be identified in the assays described above.

In addition, compounds can be identified that modulate Mrg activity by increasing or decreasing expression of an Mrg polypeptide. In a particular embodiment, agents are identified that increase Mrg activity by increasing expression of the Mrg polypeptides. These agents may include, but are not limited to nucleic acids, peptides, peptide mimetics, and small organic molecules. Agents that modulate Mrg expression, particularly those that increase expression of MrgX2, may be useful therapeutically, such as in the treatment of melanoma. Methods for determining the amount of expression of a polypeptide of interest are well known in the art.

In one embodiment the relative amounts of expression of an Mrg polypeptide are compared between a cell population that has been exposed to an agent to be tested and a control cell population. Preferably MrgX2 expression is compared, in a populations of cells known to express the Mrg polypeptide, such as WM1205LU. Expression may be detected at the nucleic acid level, such as by measurements of mRNA, or at the protein level, such as by using antibody probes.

Computer modeling and searching technologies permit identification of compounds, or the improvement of already identified compounds, that can modulate Mrg receptor expression or activity. Once an agonist or antagonist is identified, the active sites or regions, such as ligand binding sites, are determined. The active site can be identified using methods known in the art including, for example, by determining the effect of various amino acid substitutions or deletions on ligand binding or from study of complexes of the relevant compound or composition with its natural ligand, such as with X-ray crystallography.

Next, the three dimensional geometric structure of the active site is determined such as by X-ray crystallography, NMR, chemical crosslinking or other methods known in the art. Computer modeling can be utilized to make predictions about the structure where the experimental results are not clear. Examples of molecular modeling systems are the CHARMm and QUANTA programs (Polygen Corporation, Waltham, Mass.). Once a predicted structure is determined, candidate modulating compounds can be identified by searching databases containing compounds along with information on their molecular structure in an effort to find compounds that have structures capable of interacting with the active site. The compounds found from this search are potential modulators of the activity of the proteins of the present invention and can be tested in the assays described above.

L. Identification of Agents Useful for Treating Melanoma.

Agents that are identified that are able to modulate the expression level and/or activity of an Mrg polypeptide, particularly MrgX2, may be investigated further to determine if they are useful in the treatment of melanoma.

In some embodiments, the effects of Mrg agonists, antagonists or compounds that modulate expression of an Mrg polypeptide are observed on melanoma cell migration, such as in a transfilter migration assay (See, e.g., Hori et al. Biochem. Biophy. Res. Comm. 286:958-963 (2001)). In a particular embodiment, the ability of identified MrgX2 agonists to inhibit melanoma cell migration is assessed.

In another embodiment the ability of agents that decrease MrgX2 expression to inhibit melanoma cell migration is assessed. An Mrg polypeptide, such as MrgB1 or MrgX2, is expressed in a melanoma cell line that does not endogenously express an Mrg polypeptide. It is determined whether expression of the Mrg polypeptide confers responsiveness to Mrg agonists, antagonists or compounds that modulate their expression. In particular, the responsiveness of the transfected cells in the calcium release assay and transwell migration assay are assessed in the presence of such compounds.

In a further embodiment, Mrg antagonists, agonists, including agonist and antagonist antibodies, as well as compounds that modulate Mrg expression, are tested in an in vivo assay. These agents are investigated to determine whether they inhibit metastasis of Mrg expressing melanoma tumor cells in an in vivo assay, such as in the nude mouse. For example, melanoma tumor cells may be implanted in nude mice. The agent to be tested is then administered to the nude mouse and the inhibition of metastasis is measured. Such assays are well known in the art.

Agents that are identified that inhibit melanoma cell migration and/or that inhibit metastasis of Mrg polypeptide expressing melanoma tumor cells are used therapeutically in the treatment of melanoma in patients.

L. Uses for Agents that modulate at Least One Activity of the Proteins.

The present invention provides methods to treat melanoma. In the therapeutic methods of the present invention a patient is administered an effective amount of a therapeutic agents, such as an Mrg agonist, Mrg antagonist, or anti-Mrg antibody.

In a particular embodiment, one or more compounds that are identified as Mrg agonists, preferably MrgX2 agonists, are administered to a patient to treat melanoma. In another embodiment compounds that increase Mrg expression, preferably MrgX2 expression, are administered to a patient to treat melanoma. Preferably the compounds have been found to melanoma cell migration and/or metastasis, as described above.

In other embodiments an antibody to an Mrg polypeptide, preferably MrgX2, is administered to a patient to treat melanoma. The antibody may be conjugated to a toxin that kills cells expressing the Mrg polypeptide, preferably melanoma cells expressing MrgX2. In another embodiment the antibody is an agonist antibody that activates MrgX2 and preferably inhibits melanoma cell migration and/or metastasis.

In further embodiments an Mrg agonist, preferably an MrgX2 agonist is used in the preparation of a medicament for the treatment of melanoma.

As used herein, a subject to be treated can be any mammal, so long as the mammal suffering or at risk of suffering from melanoma. The invention is particularly useful in the treatment of human subjects.

The agents of the present invention can be provided alone, or in combination with other agents that modulate a particular biological or pathological process. For example, an agent of the present invention can be administered in combination with other known drugs for the treatment of melanoma. As used herein, two or more agents are said to be administered in combination when the two agents are administered simultaneously or are administered independently in a fashion such that the agents will act at the same time.

The agents are administered to a mammal, preferably to a human patient, in accord with known methods. Thus the agents of the present invention can be administered via parenteral, subcutaneous, intravenous, intramuscular, intraperitoneal, intracerebrospinal, intra-articular, intrasynovial, intrathecal, transdermal, topical, inhalation or buccal routes. They may be administered continuously by infusion or by bolus injection. Generally, where the disorder permits the agents should be delivered in a site-specific manner. Alternatively, or concurrently, administration may be by the oral route. The dosage administered will be dependent upon the age, health, and weight of the recipient, kind of concurrent treatment, if any, frequency of treatment, and the nature of the effect desired.

The toxicity and therapeutic efficacy of agents of the present invention can be determined by standard pharmaceutical procedures in cell cultures or experimental animals. While agents that exhibit toxic side effects can be used, care should be taken to design a delivery system that targets such compounds to the desired site of action in order to reduce side effects.

While individual needs vary, determination of optimal ranges of effective amounts of each component is within the skill of the art. For the prevention or treatment of melanoma, the appropriate dosage of agent will depend on a variety of factors including the particular type of melanoma to be treated, the severity and course of the disease, whether the agent is administered for preventive or therapeutic purposes, previous therapy, the patient's clinical history and response to the agent, and the discretion of the attending physician. Therapeutic agents are suitably administered to the patient at one time or over a series of treatments. Typical dosages comprise 0.1 to 100 μg/kg body wt. The preferred dosages comprise 0.1 to 10 μg/kg body wt. The most preferred dosages comprise 0.1 to 1 μg/kg body wt. For repeated administrations over several days or longer, depending on the condition, the treatment is sustained until a desired suppression of disease symptoms occurs. The progress of this therapy is easily monitored by conventional techniques and assays.

In addition to the pharmacologically active agent, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell. The agent can also be prepared as a sustained-release formulation, including semipermeable matrices of solid hydrophobic polymers containing the protein. The sustained release preparation may take the form of a gel, film or capsule.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination with other therapeutic or diagnostic agents. In certain preferred embodiments, the compounds of this invention may be co-administered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro. When used in vivo, the compounds must be sterile. This is readily accomplished by filtration through sterile filtration membranes.

a. Articles of Manufacture

In another embodiment of the invention, an article of manufacture containing materials useful for the treatment of melanoma is provided, preferably an Mrg agonist, more preferably an MrgX2 agonist. The article of manufacture comprises a container and a label or package insert(s) on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is an MrgX2 agonist. The label or package insert indicates that the composition is used for treating the condition of choice, such as to treat melanoma. In one embodiment, the label or package inserts indicates that the composition comprising the Mrg agonist can be used to treat melanoma.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

### Expression of mMrgB1

Expression of members of the MrgB subfamily of G protein coupled receptors is investigated in dorsal root ganglia. In situ hybridization was carried out using probes for mMrgB4 and mMrgB5. As shown in FIG. 2, expression of mMrgB4 and mMrgB5 was seen in subsets of dorsal root ganglia. Expression of other members of the MrgB subfamily was investigated using degenerate RT-PCR. None of the other MrgB genes were expressed in dorsal root ganglia.

In contrast to mMrgB4 and mMrgB5, mMrgB1 (SEQ ID NO: 2) was found to be specifically expressed in the skin and spleen of mice at birth. Newborn (P0) C57BL/6 (pigmented) mouse sections were hybridized with an mMrgB1 riboprobe. mMrgB1 expression was detected in scattered cells of the skin, spleen and submandibular gland, as shown in FIG. 3.

The expression of mMrgB1 was investigated further. In particular, the relationship of mMrgB1 expression to melanocyte specific markers was compared. Newborn (P0) and one week old (P9) C57/BL/6 (pigmented) mouse sections were hybridized with mMrgB1, dopachrome tautomerase (DCT)/tyrosinase-related protein 2 and Mitf riboprobes. DCT and Mitf are melanocyte markers whose expression is restricted to the developing hair follicles. MrgB1 is expressed in the lower dermis but not in the hair follicle, as shown in FIG. 4. In addition, expression of mMrgB1 in the skin is dramatically reduced as the animal ages from P1 to P9 (data not shown).

### Example 2

### Expression of hMrgX2

The expression pattern of human MrgX2 was investigated. Briefly, total RNA (approximately 20 μg) from various human Wistar Melanoma (WM) cell lines was probed with hMrgX2. The Wistar Melanoma cell lines were isolated from melanomas at different stages of progression, including radial growth phase (RGP), vertical growth phase (VGP), and metastasis (Hsu et al. “Melanoma: The Wistar Melanoma (WM) Cell Lines” pp 259-274 in Human Cell Culture, Vol. 1, 1999, J. R. W. Masters adn B. Palsson, eds.). WM1205Lu is the most highly aggressive and metastatic cell line in the Wistar collection. It was isolated from a lung metastasis in mice after injecting the WM793 cell line into the skin. The WM793 and WM1205Lu are considered matched pairs.

The WM793, WM1205Lu and WM793 cell lines were all found to express hMRGX2. Expression of hMrgX2 was also investigated in a panel of 12 different human tissues. As indicated in Table 2 below, expression of hMrgX2 was only seen in melanoma cells. For this reason, expression of hMrgX2 in epidermal cells is predicative for cancer.

TABLE 2
Expression of hMrgX2

| | |
|---|---|
| Melanoma Cell Line WM3211 | Yes |
| Melanoma Cell Line WM793 | Yes |
| Melanoma Cell Line WM1205Lu | Yes |
| Human Dorsal Root Ganglia | No |
| Human Brain | No |
| Human Heart | No |
| Human Skeletal Muscle | No |
| Human Colon | No |
| Human Thymus | No |
| Human Spleen | No |
| Human Kidney | No |
| Human Liver | No |
| Human Small Intestine | No |
| Human Placenta | No |
| Human Lung | No |
| Human Peripheral Blood Leukocytes | No |

### Example 3

### Diagnosis of Melanoma Using Antibodies to hMrgX2

A tissue sample is removed from a patient suspected of suffering from melanoma or at an elevated risk of developing the disease. For example, a mole or other skin lesion identified by a physician as potential melanoma is removed from a human patient. After appropriate preparation, the sample is contacted with an antibody to hMrgX2, preferably a monoclonal antibody. Specific binding of the antibody to the sample indicates that the patient is suffering from melanoma.

### Example 4

### Diagnosis of Melanoma Using Nucleotide Probes to hMrgX2

A tissue sample, preferably a skin sample, is removed from a patient suspected of suffering from melanoma or at an elevated risk of developing the disease. After appropriate preparation, the sample is contacted with a labeled nucleic acid probe that is able to specifically bind hMrgX2 mRNA. Specific binding of the probe to the sample indicates that the patient is suffering from melanoma.

### Example 5

### Treatment of a Patient Suffering From Melanoma

A patient is diagnosed as suffering from melanoma. An MrgX2 agonist, preferably an agonist antibody to hMrgX2, is administered to the patient according to a regimen decided upon by the attending physician based on such factors as the stage of the disease, the general health of the patient, other coincident treatments and the physicians experience. Following administration of an antibody specific for bMrgX2, the amount of melanoma in the patient is reduced.

Although the present invention has been described in detail with reference to examples above, it is understood that various modifications can be made without departing from the spirit of the invention. Accordingly, the invention is limited only by the following claims. All cited patents, patent applications and publications referred to in this application are incorporated by reference in their entirety.

What is claimed is:

1. A method of diagnosing skin cancer in a patient comprising:
providing a tissue sample from a patient suspected of having skin cancer; and
determining whether cells in said tissue sample express MrgX2, wherein expression of MrgX2 indicates that the patient has skin cancer.

2. The method of claim 1, wherein the skin cancer is melanoma.

3. The method of claim 1, wherein the tissue sample comprises skin cells.

4. The method of claim 1, wherein MrgX2 expression is determined by contacting the sample with a nucleic acid probe that is complementary to a portion of the MrgX2 nucleic acid of SEQ ID NO: 3 and determining if the probe binds to the tissue sample.

5. The method of claim 9, wherein the probe is detectably labeled.

6. The method of claim 1, wherein said MrgX2 has the amino acid sequence of SEQ ID NO: 4

7. A method of diagnosing melanoma in a patient comprising:
obtaining a tissue sample from the patient;
preparing RNA from the tissue sample;
contacting the RNA with a nucleotide probe that is capable of hybridizing to the MrgX2 nucleotide sequence of SEQ ID NO: 3 under stringent conditions; and
determining binding of the nucleotide probe to RNA in the sample.

8. The method of claim 12, wherein the nucleotide probe is detectably labeled.

9. The method of claim 12, wherein the RNA is mRNA.

10. The method of claim 12, wherein the RNA is total RNA.

11. The method of claim 12, wherein said tissue sample comprises skin cells.
